(19) United States
(12) Patent Application Publication
(10) Pub. No.: US 2014/0051988 A1
(43) Pub. Date: Feb. 20, 2014

(54) METHOD AND APPARATUS FOR REPRESENTING A BIOLOGICAL HOLLOW ORGAN

(71) Applicant: Stefan Lautenschläger, Hausen (DE)

(72) Inventor: Stefan Lautenschläger, Hausen (DE)

(21) Appl. No.: 13/966,344

(22) Filed: Aug. 14, 2013

(30) Foreign Application Priority Data
Aug. 16, 2012 (DE) 102012214589.3

(51) Int. Cl. A61B 5/06

(52) U.S. Cl. CPC A61B 5/061
USPC 600/424

(57) ABSTRACT

A method for representing a biological hollow organ is proposed. A 3D data record and a 2D data record are received. The 2D data record includes a part of a medical instrument, which is arranged within the biological hollow organ and has a point which can be detected in the 2D data record. The position of the detectable point is determined in the 2D data record and is transferred into the 3D data record. A subvolume of the 3D data record is determined. The center of gravity of the volume of the subvolume is the specific position of the detectable point in the 3D data record and the dimensions and the alignment of the subvolume can be predetermined. The subvolume of the 3D data record is visualized.

### CROSS REFERENCE TO RELATED APPLICATIONS

This application claims priority to German application No. 10 2012 214 589.39 DE filed Aug. 16, 2012, the entire content of which is hereby incorporated herein by reference.

### FIELD OF INVENTION

The present invention relates to a method for representing a biological hollow organ. Furthermore, the present invention relates to a corresponding apparatus for representing a biological hollow organ.

### BACKGROUND OF INVENTION

In clinical practice, for instance during diagnosis or a surgical intervention, imaging methods, such as computed tomography, magnetic resonance tomography or x-ray imaging, are increasingly being used. Particularly with catheter-based interventions, such as the treatment of arteriovenous malformation, AVM, or the treatment of an aneurysm, it is important here for a treating physician to obtain a representation of the position of a medical catheter within a vascular tree and relative to structures surrounding the same for instance, as a result of which the orientation in a possibly complex vascular tree is facilitated. The superimposition of a “live” image, i.e. an image which reproduces the momentary state, for instance a radioscopy or fluoroscopic image, with a previously recorded spatial image of the vascular tree may be helpful. With current image systems, it is disadvantageous that the spatial representation during superimposition is projected onto the “live” image and thus part of the spatial information can get lost. Therefore superimposed or overlapping structures, such as superimposed vessels, can often only be identified with difficulty or not at all. Important structures, such as an aneurysm to be treated, of vessels, which are found in the beam path of the imaging apparatus upstream of the structure of interest, can also be concealed. It may happen in this situation that an aneurysm is overlooked for instance or a new projection setting must be sought in a time-consuming fashion and with additional radiation exposure, from which the aneurysm can be identified.

### SUMMARY OF INVENTION

The object of the present invention now consists in specifying a method for representing a biological hollow organ, which does not feature the described disadvantages. The object of the invention is further to describe a corresponding apparatus.

The invention achieves this object with a method for representing a biological hollow organ having the features of the first independent claim and an apparatus for representing a biological hollow organ having the features of the second independent claim.

The basic idea behind the invention is a method for representing a biological hollow organ, including the following method steps:
S1) Receiving a 3D data record, which includes an examination area of an examination object;
S2) Receiving a 2D data record, which includes the examination area of the examination object and at least one part of a medical instrument, wherein the at least one part of the medical instrument is arranged within a biological hollow organ and comprises a point which can be detected in the 2D data record;
S3) Determining the position of the detectable point of the at least one part of the medical instrument in the 2D data record;
S4) Transferring the specific position of the detectable point of the at least one part of the medical instrument in the 2D data record into the 3D data record;
S5) Determining a subvolume of the 3D data record, wherein the center of gravity of the volume of the subvolume is the specific position of the detectable point of the at least one part of the medical instrument in the 3D data record and the dimensions and the alignment of the subvolume can be predetermined;
S6) Visualizing the subvolume of the 3D data record.

The inventive method allows for the representation of a biological hollow organ. A biological hollow organ is understood to be an organ which encloses a hollow cavity, also known as lumen, with its biological tissue. The hollow organs include for instance esophagus, gastrointestinal tract, gallbladder, windpipe, heart, the female genital tract with fallopian tubes, womb, vagina, the urinary tract with renal pelvis, ureter, urinary bladder and urethra. Vessels belonging to the hollow organs should in particular also be included here. Vessels are blood vessels such as arteries, veins, capillaries and lymph vessels such as lymph capillaries, collectors and lymphatic collecting vessels. The guidance of a medical instrument in a hollow organ, e.g. from the esophagus into the stomach, is advantageous in that the medical instrument can be advanced in a defined manner relatively simply in most instances since the path through the natural course of the hollow organ is predetermined.

In the first method step, a 3D data record, which includes an examination area of an examination object, is received, acquired, obtained or loaded into a memory of a computing and control means, e.g. of a computer. The examination object may be a person or an animal. A 3D data record, which represents a spatial image, can be obtained by computed tomography (CT), magnetic resonance tomography (MR) or another spatial imaging method. A further option consists in using an x-ray device. In accordance with the prior art, a spatial image can be calculated by means of a suitable series of x-ray recordings from different directions around the examination object. The 3D data record includes at least one examination area, which includes in particular the biological hollow organ. For instance, the examination area may be the stomach region for instance and the biological hollow organ is a blood vessel in the region of the stomach. The 3D data record can preferably be segmented or is already segmented with the aid of a segmentation algorithm for instance. Segmentation is a common method in medical image processing. In this context, the term “release an anatomical object from surrounding tissue, bones and otherwise” cannot be understood as image components associated with the anatomical object. The anatomical object is for instance an organ or a vessel, which is included in the examination area. A simple segmentation method is for instance a threshold value method.

In the second method step, a 2D data record is received, acquired, obtained or loaded into a memory of a computing and control means, e.g. of a computer. The 2D data record includes the examination area of the examination object and at least one part of a medical instrument, wherein the at least one part of the medical instrument is arranged within a biological hollow organ and has a point which can be detected in the 2D data record. In clinical practice, two-dimensional images of a region of interest are frequently recorded during diagnostic or surgical procedures, since by comparison with spatial images they signify in most instances a lower radiation exposure to the patient and/or can be obtained more easily and/or more quickly. A 2D data record represents a two-dimensional image, which was recorded with an imaging method, for instance with the aid of an x-ray device. The 2D data record includes the examination area of the examination object, for instance the stomach region of a human patient. Furthermore, the 2D data record includes at least one part of a medical instrument, which is arranged within a biological hollow organ and which comprises a point which can be detected in the 2D data record. This means that part of the medical instrument, e.g. a surgical scalpel, is found within a biological hollow organ, e.g. of a blood vessel and that a point in part of the medical instrument can be detected in the 2D data record. The term detection is understood to mean “identify”, “trace”, discover” or “discern”. The detectability may be provided in that a marker, which is arranged on the part of the medical instrument, is visible for instance on an x-ray image, or that the part of the medical instrument comprises a striking point or a marked structure, for instance a surrounding groove, which can be detected in the 2D data record.

In the third method step, the position of the detectable point of the at least one part of the medical instrument is determined in the 2D data record. The detectability of the point on the part of the medical instrument allows the position of this point to be determined in the 2D data record. The position can be specified for instance by a local vector of a Cartesian coordinate system of the 2D data record in respect of the detected point of the medical instrument.

In the fourth method step, the specific position of the detectable point of the at least one part of the medical instrument in the 2D data record is transferred or transformed into the 3D data record. Since both the 2D data record and also the 3D data record include the examination area of the examination object, the specific position of the detectable point of the at least one part of the medical instrument specified in the 2D data record is also contained in the 3D data record. In order to determine the position of the detectable point in the 3D data record, it would be conceivable for instance to register the 2D data record in the 3D data record. Registration is understood to mean a method involving digital image processing, which is used to harmonize two images with one another as best possible. Since this embodiment is a 2D data record and a 3D data record, a 2D-3D registration is needed. Image registration in medical image processing is a frequently used method, for which a plurality of image registration algorithms is known from the prior art. The aim is to calculate a transformation, which adjusts an image, known as object image, in the best way possible to another image, known as reference image. This is therefore an optimization task, in which a quality criterion, e.g. a deviation, is to be minimized or a consistency or similarity is to be maximized. If a transformation is found, which describes the position of the 2D data record in the 3D data record, the position of the detectable point of the at least one part of the medical instrument in the 3D data record is known e.g. as the local vector of a Cartesian coordinate system of the 3D data record with respect to the detected point of the medical instrument. The information that the detectable point is found within the biological hollow organ, e.g. a blood vessel is advantageously included in the determination of the position of the detectable point of the at least one part of the medical instrument in the 2D data record into the 3D data record. The possibilities are therefore restricted to positions within the biological hollow organ. A probability mask is further advantageously included in the determination of the position of the detectable point of the at least one part of the medical instrument in the 2D data record into the 3D data record. The probability mask includes probability values, which describe the probability of a deflection, stretching or general change in shape of the biological hollow organ.

A subvolume of the 3D data record is determined in the fifth method step from the 3D data record. The center of gravity of the volume of the subvolume is provided by the specific position of the detectable point of the at least one part of the medical instrument in the 3D data record. The dimensions and alignment of the subvolume can be predetermined. The subvolume is thus a sub quantity of the 3D data record. The shape, dimensions and if necessary alignment of the subvolume can be predetermined. The position of the subvolume within the 3D data record is as a result determined in that the center of gravity of the volume of the subvolume comes to rest on the specific position of the detectable point of the at least one part of the medical instrument in the 3D data record. It is therefore conceivable that the shape of the subvolume is predetermined as a cylinder for instance by an operating person. Furthermore, the dimensions are predetermined with a length of 10 cm and a diameter of 3 cm and the alignment such that the longitudinal axis of the cylinder is aligned with the longitudinal axis of a blood vessel. The center of gravity of the volume of the cylinder is placed on the specific position of the detectable point of the at least one part of the medical instrument in the 2D data record transferred into the 3D data record. The subvolume of the 3D data record is thus determined clearly in the 3D data record.

In the sixth method step, the subvolume of the 3D data record is visualized.

Since the 3D data record represents a spatial image, which can be shown for instance on a representation means, the subvolume of the 3D data record can also be shown as a spatial image or as a projection of a spatial image.

The medical instrument is preferably a catheter or a guide wire, and the point which can be detected in the 2D data record is the tip of the medical instrument.

A medical catheter or, in brief, catheter is understood in particular to mean a hose or rod-type device with a length of approx. 0.3 to 1.5 m and a diameter of approx. 1 to 20 mm, which can be inserted into a human or animal body, in particular in vessels. A medical catheter can also include instruments which are integrated or can be inserted by way of working channels, e.g. micro mechanical devices, such as small clamps or grippers, with which examining or intervening processes can be implemented. A guide wire is a wire-type device with a length of approximately 0.3 to 1.5 m, which can likewise be inserted into a human or animal body, in particular into vessels. The tip of a catheter or a guide wire is in most instances easily identifiable in a 2D image and thus lends itself to be the detectable point in the medical instrument. The surroundings of the tip of a catheter or a guide wire is often of particular interest to a user, e.g. a physician. These surroundings are visualized in the sixth method step with this embodiment of the inventive method.

The 2D data record is favorably a radioscopy image.

Radioscopy or fluoroscopy is understood in radiology or x-ray diagnostics to mean the continual observance of processes in the human or animal body using x-rays. A radioscopy image is an image from a series of image which were obtained during a radioscopy or fluoroscopy. In particular, the last image of a radioscopy, i.e. the temporally most up-to-date is of particular importance to a radiologist for instance.

The subvolume of the 3D data record is particularly advantageously a ball and the radius of the ball can be predetermined.

When using a spherical shape as the shape of the subvolume of the 3D data record in the fifth method step, the necessary prespecifications reduce to the dimensions, in this case the radius, of the subvolume. The center of gravity of the volume is the central point of the ball, the alignment is irrelevant. A constant value can be predetermined for instance as a radius.

It has proven to be advantageous if the subvolume of the 3D data record is a ball and the radius of the ball is predetermined by the following method step:
S51) Receiving a factor;
S52) Determining a radius of the biological hollow organ at the specific position of the detectable point of the at least one part of the medical instrument in the 3D data record;
S53) Calculating the radius of the ball by multiplying the radius of the biological hollow organ at the specific position of the detectable point with the received factor.

These method steps describe a particularly advantageous procedure for determining or prespecifying the radius of a spherical subvolume of the 3D data record. The idea underlying the method is to prespecify the radius of the spherical subvolume to be visualized as a function of the radius of the biological hollow organ. To this end, a factor, preferably greater than one, e.g. two or five, is required, which is predetermined for instance by an operating person on an input means. The radius of the biological hollow organ is then determined at the detectable point of the medical instrument in the 3D data record. The radius of the ball results from the product of the received factor and the radius of the biological hollow organ. If the radius of the biological hollow organ at the detectable point of the medical instrument in the 3D data record is not clearly determinable because the biological hollow organ at the detectable point has a number of possible radii for instance, it is conceivable for the radius to be used to be specified for instance by an operating person. If the cross-section of the biological hollow organ at the detectable point is not circular, a central radius can expediently be used.

In an advantageous development of the invention, a binary 3D mask is included in the determination of the subvolume of the 3D data record, the center of gravity of the volume of which is the specific position of the detectable point of the at least one part of the medical instrument in the 3D data record and the dimensions of which can be predetermined. A multiplication of the binary 3D mask with the 3D data record is included in the determination of the subvolume of the 3D data record.

In this development, the subvolume of the 3D data record is determined by multiplying a binary 3D mask with the 3D data record. The shape, dimensions and if necessary alignment of the binary 3D mask can be predetermined. The position of the binary 3D mask within the 3D data record is determined in that the center of gravity of the volume of the binary 3D mask comes to rest at the specific position of the detectable point of the at least one part of the medical instrument in the 3D data record, i.e. the center of gravity of the volume of the binary 3D mask is provided by the specific position of the detectable point of the at least one part of the medical instrument in the 3D data record. The shape of the binary 3D mask is advantageously a ball and the radius of the ball can be predetermined. It is further advantageous to predetermine the radius of the spherical, binary 3D mask as a function of the radius of the biological hollow organ at the specific position of the detectable point of the at least one part of the medical instrument in the 3D data record. The elements of the binary 3D mask may comprise a first value, for instance the binary value “1”, if they lie within or on the predetermined volume shape, such as for instance the ball, otherwise they have a complementary value, e.g. the binary value “0”. By multiplying the binary 3D mask with the 3D data record, the subvolume of the 3D data record of interest, which is visualized, is obtained.

In a further advantageous embodiment of the invention, the visualization of the subvolume of the 3D data record includes a superimposition of the 2D data record with a correctly positioned projection of the subvolume of the 3D data record.

As described in the introduction, current imaging systems are disadvantageous in that a spatial representation during superimposition is projected onto a “live” image and a part of the spatial information can thus get lost, as a result of which superimposed or overlapping structures, such as superimposed vessels, can often only be recognized with difficulty or not at all. By means of one of the inventive methods, the entire 3D data record is no longer superimposed onto a 2D image, but instead the subvolume of the 3D data record, which essentially only includes the region of interest.

A further advantageous embodiment provides that the visualization of the subvolume of the 3D data record includes an additional representation of the subvolume of the 3D data record with a predeterminable projection angle.

In this embodiment, the subvolume of the 3D data record is also shown for instance on a means of representation, such as monitor. The projection angle or angle of rotation and/or the axis of rotation of the subvolume can be predetermined. It is therefore conceivable to represent additional representations of the subvolume of the 3D data record, e.g. from orthogonal directions of view, in respect of superimposing a 2D “live” image with the positionally correct subvolume of the 3D data record.

Furthermore, further improvements can be achieved if the method is executed automatically.

Automatically executed methods require little user input and thus result in a quicker work flow. Since current imaging apparatuses are in most instances already prepared for automatic work flows, the integration of an inventive method is technically easily possible. Furthermore, automatically executed methods are often less prone to faults.

A further basic idea behind the invention relates to an apparatus for representing a biological hollow organ. The apparatus includes a computing and control means, which is configured to execute the following method steps:
S1) Receiving a 3D data record, which includes an examination area of an examination object;
S2) Receiving a 2D data record, which includes the examination area of the examination object and at least one part of a medical instrument, wherein the at least one part of the medical instrument is arranged within a biological hollow organ and comprises a point which can be detected in the 2D data record;
S3) Determining the position of the detectable point of the at least one part of the medical instrument in the 2D data record;
S4) Transferring the specific position of the detectable point of the at least one part of the medical instrument in the 2D data record into the 3D data record;
S5) Determining a subvolume of the 3D data record, wherein the center of gravity of the volume of the subvolume is the specific position of the detectable point of the at least one part of the medical instrument in the 3D data record and the dimensions and the alignment of the subvolume can be predetermined.

Furthermore, the apparatus includes a means of representation for visualizing the subvolume of the 3D data record.

The inventive apparatus thus includes at least one computing and control means, e.g. a computer, and a display means, e.g. a computer monitor. The computing and control means is embodied to as to receive a 3D data record, which includes an examination area of an examination object, and a 2D data record, which includes the examination area of the examination object and at least one part of a medical instrument. This can be realized for instance by a data interface, with the aid of which it can receive an x-ray image from an x-ray device. The computing and control means is further embodied so as to execute the described image processing tasks, by it making available for instance a suitable computer program, in particular from the field of medical, digital processing, and operating the same. The subvolume of the 3D data record can be visualized on the display means, e.g. by representation on the computer monitor.

A preferred embodiment of a basic idea of the invention provides that the apparatus is configured

The ability to execute the described method steps can herewith also be realized by corresponding computer programs, which can be called up in the computing and control unit.

### BRIEF DESCRIPTION OF THE DRAWINGS

The exemplary embodiments described in more detail below represent preferred embodiment variants of the present invention. Further advantageous developments result from the subsequent figures and the description. In the figures:

FIG. 1 shows by way of example a flow chart of an inventive method for representing a biological hollow organ;

FIG. 2 shows by way of example an image of a 3D data record of a vascular tree according to the prior art;

FIG. 3 shows by way of example an image of a 2D data record of a vascular tree according to the prior art;

FIG. 4 shows by way of example an image of a 2D-3D superimposition of a vascular tree according to the prior art;

FIG. 5 shows an exemplary embodiment of an inventive representation of a vascular tree;

FIG. 6 shows a schematic representation of a 3D data record having a spherical subvolume of the 3D data record;

FIG. 7 shows a schematic representation of an exemplary embodiment of an inventive apparatus for representing a biological hollow organ.

### DETAILED DESCRIPTION OF INVENTION

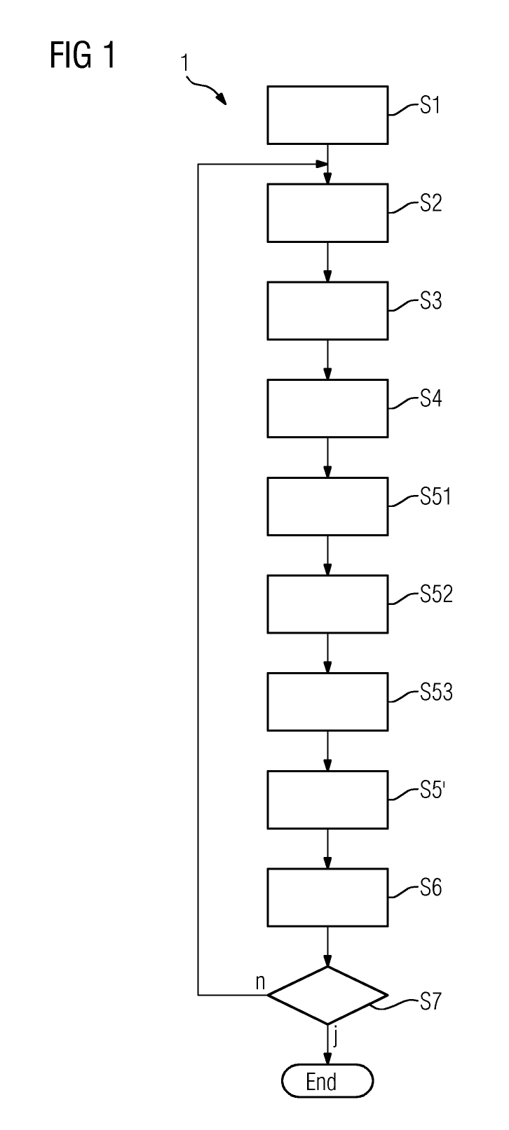

FIG. 1 shows an exemplary embodiment of a flow chart of an inventive method 1 for representing a biological hollow organ. The method 1 includes the methods steps S1 to S4, S51 to S53, S5′ and S6 to S7. It begins with methods step S1 and ends “End”, after method step S7. The individual methods steps read:
S1) Receiving a 3D data record, which includes an examination area of an examination object;
S2) Receiving a 2D data record, which includes the examination area of the examination object and at least one part of a medical instrument, wherein the at least one part of the medical instrument is arranged within a biological hollow organ and comprises a point which can be detected in the 2D data record;
S3) Determining the position of the detectable point of the at least one part of the medical instrument in the 2D data record;
S4) Transferring the specific position of the detectable point of the at least one part of the medical instrument in the 2D data record into the 3D data record;
S51) Receiving a factor;
S52) Determining a radius of the biological hollow organ at the specific position of the detectable point of the at least one part of the medical instrument in the 3D data record;
S53) Calculating a radius of the ball by multiplying the radius of the biological hollow organ at the specific position of the detectable point with the received factor.
S5′) Determining a spherical subvolume of the 3D data record with the radius of the ball from method step S53, wherein the central point of the subvolume is the specific position of the detectable point of the at least one part of the medical instrument in the 3D data record;
S6) Visualizing the subvolume of the 3D data record.
S7) Testing an abort criterion, if the abort criterion is fulfilled, End “Y”, “End” the method 1, if the abort criterion is not fulfilled, “N”, Jump to method step S2.

It is advantageous if an abort criterion, S7, is checked after method step S6. An abort criterion can be understood to mean for instance the reaching of a predeterminable number of method processes or the actuation of a button, i.e. if for instance the button is pressed, the abort criterion is fulfilled, “Y” and the method 1 is ended. If the abort criterion is not fulfilled “N”, a further radioscopy image is branched to method step S1, “Receiving a 2D data record”. The visualization of the subvolume of the 3D data record in method step S6 preferably includes a superimposition of the 2D data record with a positionally correct projection of the subvolume of the 3D data record. Furthermore, the visualization of the subvolume of the 3D data record in method step S6 preferably includes an additional representation of the subvolume of the 3D data record with a predeterminable projection angle. The method 1 is preferably executed automatically, i.e. for instance the position of the detectable point of the at least one part of the medical instrument is automatically determined in the 2D data record after receiving the 3D data record and 2D data record, the following method steps are executed automatically and the subvolume of the 3D data record is then visualized. This allows for the quickest possible implementation of the imaging and the minimal possible exposure in terms of time to the patient.

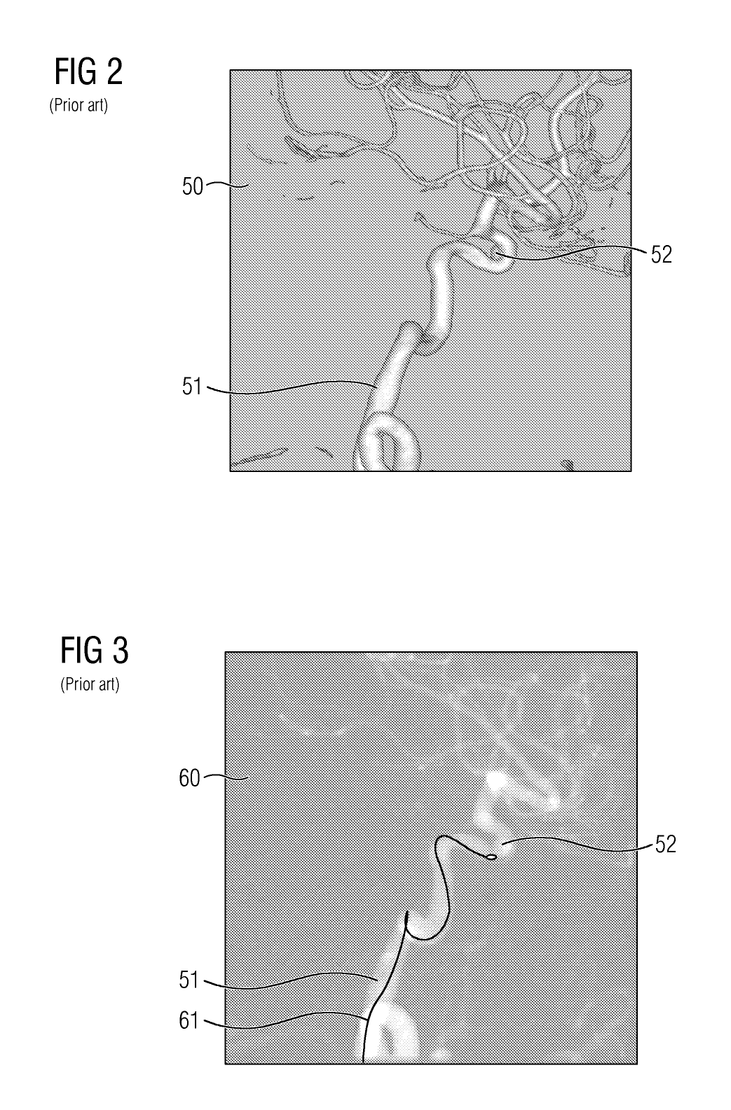

In FIG. 2, an image of a 3D data record 50 of a biological hollow organ 51, here a vascular tree, is shown by way of example in accordance with the prior art. Since the 3D data record 50 was obtained prior to a catheter-assisted examination using a computed tomography device, no medical catheter is contained in the image. In a winding of the vascular tree, an aneurysm 52, in other words a diseased, locally bounded, frequently sack-like extension, can be identified.

In FIG. 3, an image of a 2D data record 60 of a biological hollow organ 51, here a vascular tree, is shown by way of example in accordance with the prior art. The 2D data record 60 was obtained during radioscopy using an x-ray device, i.e. a radioscopy image exists, which was obtained from a series of images during radioscopy or fluoroscopy. In a winding of the vascular tree, an aneurysm 52 can be identified, wherein the clarity of the representation is less good than in the case of an aneurysm of the image of the 3D data record in FIG. 2. An important advantage in the use of 2D representations is that recordings can be made more frequently thereby producing the impression of “live” images. Continuous recordings are then important if for instance the movement of a medical instrument 61, such as in this exemplary embodiment a catheter, is to be monitored.

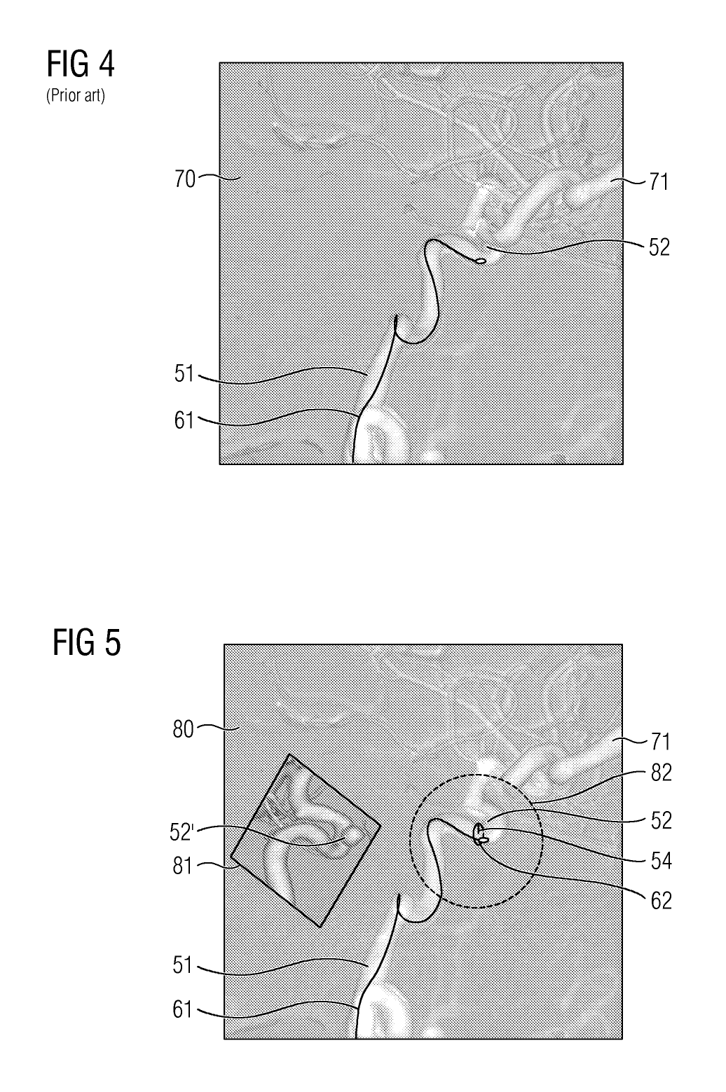

In FIG. 4, an image of a 2D-3D superimposition 70 of a biological hollow organ 51, here a vascular tree, is shown by way of example in accordance with the prior art. The superimposition, in which a projection of a spatial image, e.g. a computed tomography image, is superimposed onto a 2D image, e.g. a fluoroscopy, is to combine the cited advantages of both forms of representation. In particular, a medical instrument 61, here a catheter, can be identified, which is guided into the plastically acting vascular tree. The images which result in superimposition equate to those from FIGS. 2 and 3, wherein in this exemplary embodiment the vascular tree and an aneurysm 52 are partially concealed by a further vessel 71. If the vessel 71 were to extend further into the center of the image, the aneurysm 52 would not be visible and the per se informative representation would not be usable for an examination or a surgical intervention. The consequence would then be to switch of this overlay mode for instance and if necessary to rotate the spatial image by means of manual input so that the aneurysm 52 is again visible. This would be a time-consuming procedure and the representation of the medical instrument 61 would also get lost.

FIG. 5 shows an exemplary embodiment of an inventive representation 80 of a biological hollow organ 51, here a vascular tree. A medical instrument 61, here a catheter, is introduced into the vascular tree, said catheter being visible in a 2D image, e.g. a fluoroscopy. A projection of a spatial image, e.g. of a computed tomography image, is superimposed onto the 2D image. In this respect, the representation from the exemplary embodiment from FIG. 4 is similar. An essential difference is however that an important area of the vascular tree, in particular the area in which an aneurysm 52 is found, is not concealed by a further vessel 71. This representation is achieved by an embodiment of an inventive method, which is described by way of example below. After receiving a 3D data record, which includes an examination area, here the vascular tree, of an examination object, e.g. a human patient, a 2D data record, which includes the examination area of the examination object and at least one part of a medical instrument 61, here the catheter, is received. The at least one part of the medical instrument 61 is arranged within a biological hollow organ 51, here the vascular tree, and comprises a point 62, which can be detected in the 2D data record, here the transition of the wire-type catheter shaft into the lens-shaped catheter tip, which can be easily identified in the 2D data record and thus also detected. In the next method steps, the position of the detectable point 62 of the at least one part of the medical instrument 61 is determined in the 2D data record and transferred into the 3D data record. The spatial position of the detectable point 62 is thus determined in the 3D data record. A factor is next received e.g. by an operating person, e.g. the factor “12”. A radius 54 of the biological hollow organ 51 at the specific position of the detectable point 62 of the at least one part of the medical instrument 61 is then determined in the 3D data record. By multiplying the radius 54 of the biological hollow organ 51 at the specific position of the detectable point 62 with the received factor, a radius of a ball is calculated. A spherical subvolume 82 of the 3D data record is determined with the radius of the ball, wherein the central point of the subvolume 82 is the specific position of the detectable point 62 of the at least one part of the medical instrument 61 in the 3D data record. The subvolume 82 of the 3D data record is finally visualized by it superimposing the same, as shown in FIG. 5, in a positionally correct manner onto the 2D image. It is apparent that only structures within the spherical subvolume are also shown in this area, other structures, such as the vessel 71, are not shown or are faded out. The aneurysm 52 is thus not concealed by the vessel 71. A conventional superimposition can be shown outside of the spherical subvolume. In FIG. 5 a further visualization 81 of a subvolume of the 3D data record is shown. A square-shaped subvolume of the 3D data record is determined, wherein the center of gravity of the volume of the square-shaped subvolume is the specific position of the detectable point 62 of the at least one part of the medical instrument 61 in the 3D data record and the dimensions and the alignment of the subvolume can be predetermined for instance by an operating person. The alignment or the projection direction were rotated by approximately 180° relative to the first visualization 82, so that the aneurysm 52 can be seen very clearly in one representation 52′ almost from the other side. This additional representation 81 of the subvolume of the 3D data record with a predeterminable projection angle is also referred to as side-by-side representation.

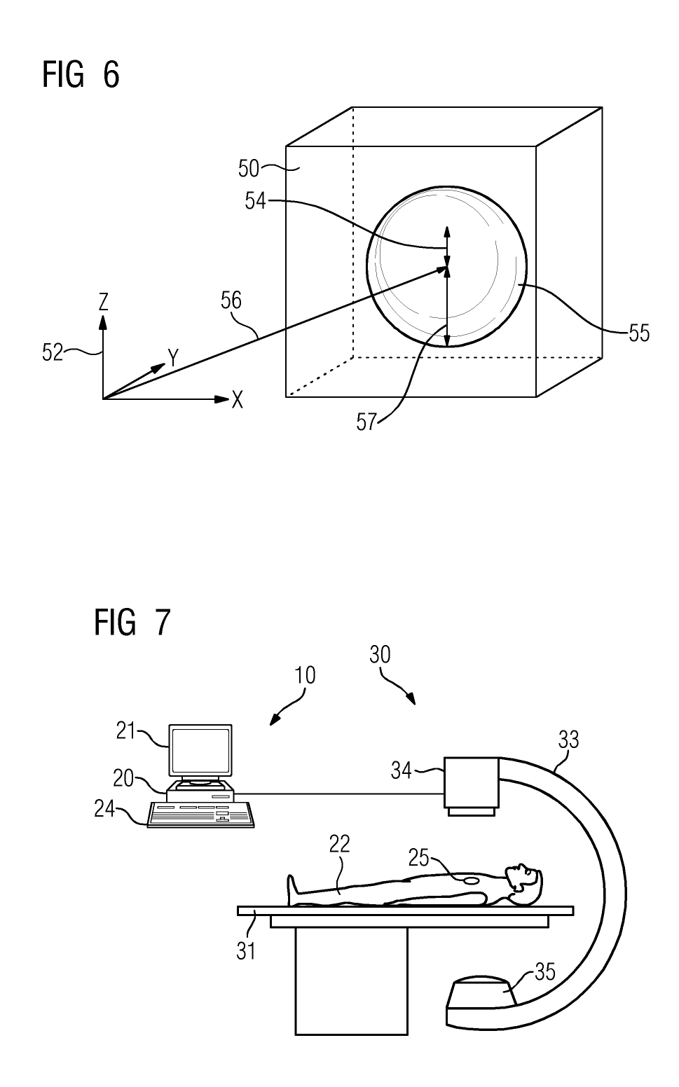

In FIG. 6 a representation of a 3D data record 50 with a spherical subvolume 55 of the 3D data record 50 is shown schematically. A local vector 56 of a Cartesian coordinate system 52 describes the specific position of a detectable point of a medical instrument in a 2D data record, transferred into the 3D data record 50. A radius 54 is the radius of a biological hollow organ at the specific position of the detectable point of the medical instrument in the 3D data record 50. The radius 57 of the spherical subvolume 55 results from the product of radius 54 with a received factor which in this instance amounts to approximately 2.

In FIG. 7 a schematic representation of an exemplary embodiment of an inventive apparatus for representing a biological hollow organ is finally shown. The apparatus 10 includes a computing and control means 20, which is configured to execute one of the described methods according to the invention. In particular, the computing and control means 20 is embodied to receive images of an x-ray apparatus 30. The x-ray apparatus 30 comprises for instance a C-arm 33, on which an x-ray source 35 and a digital x-ray detector 34 are arranged in opposing positions. An examination object 22, for instance a human patient, rests on a support facility 31, here an examination couch, which is held by a column of the support facility. The x-ray apparatus 30 can be controlled by the computing and control means 20, here a computer. The C-arm 33 of the x-ray apparatus 30 is rotatable. In accordance with the prior art, a spatial image of an examination area 25, here the breast area of the examination object 22, can be calculated and transferred to the computing and control means 20 by means of a suitable series of x-ray recordings from different directions around the examination object 22. Furthermore, the x-ray apparatus 30 can record radioscopy images of the examination area 25 and transfer them to the computing and control means 20. Entries, such as entering a factor or a projection angle, can be performed on an entry means 24, here a computer keyboard. The representation of a biological hollow organ of the examination area 25 of the examination object 22 can take place on a means of representation 21, here a computer monitor.

In summary, a further exemplary embodiment and some advantages of embodiments of the invention are described by way of example:

If complex catheter-based interventions are implemented, it is extremely important for the executing person to have the orientation within a vascular tree. Present-day software instruments assist a physician but have until now only provided the possibility of representing a previously obtained spatial image in a projection direction, in most instances in the direction of a used C-arm of an x-ray device. One further difficulty is that the spatial information is mapped onto a current “live” image, e.g. during fluoroscopy as a 2D projection, as a result of which information can get lost. There is the risk here that this is insufficient for a projection direction if two vessels overlap in the space or if an object of interest, e.g. an aneurysm, lies “behind” a vessel. In this case, the aneurysm can only be seen with difficulty and an, as proposed, additional “lateral” view would be advantageous in order to obtain the complete spatial information. With one embodiment of the invention, a catheter or a guide wire is automatically detected in a radioscopy image, known as tracking. In a computing and control unit, e.g. a computer, the catheter or guide wire position is transformed into a 3D coordinate system of a previously acquired 3D image, e.g. of a CT, MR or rotation x-ray device. The transformation is assisted here by the information that the catheter or the guide wire can only lie within a vessel, wherein the vessel is also part of the 3D image. One possible expansion of vessels can be compensated for, for instance, by using a per se known probability mask of possible bends or changes in the vessel. Furthermore, the position of the catheter or guide wire tip is detected and transformed into the 3D coordinate system of the 3D image. The radius of the vessel at this position is determined and is then multiplied with a prior received factor, which was entered or predetermined for instance by an operating person. The product is interpreted as the radius of a ball, the central point of which lies on the transformed position of the catheter or guide wire tip. The position of the ball and its extension is regarded as a new 3D volume, wherein the dimensions of the new 3D volume equate to those of the 3D image. A binary information “1” is assigned to all spatial pixels, also known as voxels, which lie within or on the ball of the new 3D volume, a binary information “0” is assigned to all voxels, which lie outside of the ball. This means that the new 3D volume is a binary volume which only includes values “0” and “1” and can also be interpreted as mask volume. In the next step, the binary volume is multiplied with the 3D image. As a result, a target volume is obtained which only includes the immediate vicinity around the catheter or guide wire tip. In other words, the target volume is a subvolume which only ever shows the immediate vicinity around the catheter or guide wire tip. The advantage is that vessels, which are further remote from the catheter or guide wire tip and are thus regarded as less relevant, are not contained in the target volume. The target volume is next used as the superimposition volume and the superimposition is displayed. This is similar to a conventional representation, with the essential difference that only relevant structures, i.e. those structures which are arranged close to the catheter or guide wire tip, are superimposed. As an alternative or additionally to the described representation, the target volume can be rotated about a predeterminable axis and a predeterminable angle, e.g. 90° or 180° and the result can be shown as an additional image, e.g. side-by-side or picture-in picture, PiP. This means that a vessel can be observed from several sides.

By superimposing an in particular real-time similar 2D image with a subvolume image of a vessel, e.g. whenever a pedal is activated, the current situation is reproduced promptly during an examination.

Since the inventive method can also be executed automatically, i.e. without user input, it is simple to handle and to control. As a result that only essential parts of the overall volume are superimposed, a user, e.g. a physician, is not distracted by insignificant information.

The possible real-time similar adjustment of the superimposition of the subvolume and the corresponding subvolume rotated in any way according to requirements in one or several additional representations allows for the orientation within the examination to be simplified significantly. Furthermore, structures, such as pathological changes, can be easily identified, if the subvolume is shown from several perspectives. The risk that these structures are overlooked because they are concealed in a conventional projection direction “behind” another object, is reduced.

The real-time similar adjustment of the superimposition increases the certainty that a medical instrument is guided to the correct position, therefore increases the safety for patients and reduces the risk of complications.

1. A method for representing a biological hollow organ, comprising:
receiving a 3D data record comprising an examination area of an examination object;
receiving a 2D data record comprising the examination area of the examination object and at least one part of a medical instrument, wherein the at least one part of the medical instrument is arranged within a biological hollow organ and comprises a point which can be detected in the 2D data record;
determining the position of the detectable point of the at least one part of the medical instrument in the 2D data record;
transferring the specific position of the detectable point of the at least one part of the medical instrument in the 2D data record into the 3D data record;
determining a subvolume of the 3D data record, wherein a center of gravity of the volume of the subvolume is the specific position of the detectable point of the at least one part of the medical instrument in the 3D data record and the dimensions and the alignment of the subvolume can be predetermined; and
visualizing the subvolume of the 3D data record.

2. The method as claimed in claim 1, wherein the medical instrument is a catheter or a guide wire and wherein the point which can be detected in the 2D data record is a tip of the medical instrument.

3. The method as claimed in claim 1, wherein the 2D data record is a radioscopy image.

4. The method as claimed in claim 1, wherein the subvolume of the 3D data record is a ball and radius of the ball can be predetermined.

5. The method as claimed in claim 1, wherein the subvolume of the 3D data record is a ball and the radius of the ball can be predetermined by:
receiving a factor;
determining a radius of the biological hollow organ at the specific position of the detectable point of the at least one part of the medical instrument in the 3D data record;
calculating the radius of the ball by multiplying the radius of the biological hollow organ at the specific position of the detectable point with the received factor.

6. The method as claimed in claim 1, wherein a binary 3D mask is included in the determination of the subvolume of the 3D data record, wherein the center of gravity of the volume of which is the specific position of the detectable point of the at least one part of the medical instrument in the 3D data record and the dimensions of which can be predetermined, and wherein a multiplication of the binary 3D mask with the 3D data record is included in the determination of the subvolume.

7. The method as claimed in claim 1, wherein the visualization of the subvolume of the 3D data record comprises a superimposition of the 2D data record with a positionally correct projection of the subvolume of the 3D data record.

8. The method as claimed in claim 1, wherein the visualization of the subvolume of the 3D data record comprises an additional representation of the subvolume of the 3D data record with a predeterminable projection angle.

9. The method as claimed in claim 1, wherein the method is executed automatically.

10. An apparatus for representing a biological hollow organ, comprising:
a computing and control device configured to execute following steps:
receiving a 3D data record comprising an examination area of an examination object;
receiving a 2D data record comprising the examination area of the examination object and at least one part of a medical instrument, wherein the at least one part of the medical instrument is arranged within a biological hollow organ and comprises a point which can be detected in the 2D data record;
determining the position of the detectable point of the at least one part of the medical instrument in the 2D data record;
transferring the specific position of the detectable point of the at least one part of the medical instrument in the 2D data record into the 3D data record;
determining a subvolume of the 3D data record, wherein a center of gravity of the volume of the subvolume is the specific position of the detectable point of the at least one part of the medical instrument in the 3D data record and the dimensions and the alignment of the subvolume can be predetermined; and
a monitor for visualizing the subvolume of the 3D data record.
